(19) United States
(12) Patent Application Publication
(10) Pub. No.: US 2017/0035366 A1
(43) Pub. Date: Feb. 9, 2017

(54) BLOOD PRESSURE RELATED INFORMATION DISPLAY APPARATUS AND PROGRAM

(71) Applicant: OMRON HEALTHCARE CO., LTD., Muko-Shi (JP)

(72) Inventors: Mitsuo KUWABARA, Kyoto (JP); Kazuomi KARIO, Tochigi (JP)

(21) Appl. No.: 15/299,798

(22) Filed: Oct. 21, 2016

Related U.S. Application Data
(63) Continuation of application No. PCT/JP2015/063289, filed on May 8, 2015.

(30) Foreign Application Priority Data
May 14, 2014 (JP) 2014-100681

(51) Int. Cl. A61B 5/00; A61B 5/022

(52) U.S. Cl. CPC A61B 5/743; A61B 5/022; A61B 5/7235; A61B 5/0004

(57) ABSTRACT

A blood pressure related information display apparatus acquires blood pressure data including at least early-morning blood pressure data measured in the morning and night-time blood pressure data measured at night in a certain period for the measurement subject. The blood pressure related information display apparatus sets one of early-morning blood pressure and night-time blood pressure on a horizontal axis and the other on a vertical axis and performs graph display of points determined using early-morning blood pressure data and night-time blood pressure data on the display screen.

### CROSS REFERENCE TO RELATED APPLICATIONS

This application claims the benefit of priority to Japanese Patent Application 2014-100681 filed May 14, 2014 and is a Continuation Application of PCT/JP2015/063289 filed on May 8, 2015. The entire contents of each application are hereby incorporated herein by reference.

### BACKGROUND OF THE INVENTION

1. Field of the Invention

The present invention relates to a blood pressure related information display apparatus that displays information related to blood pressure of a measurement subject on a display screen.

Also, the present invention relates to a program for causing a computer to execute a method for displaying information related to blood pressure of a measurement subject on a display screen.

2. Description of the Related Art

Conventionally, as an apparatus that displays information related to blood pressure of a measurement subject, as disclosed in JP 2004-261452A, there is known to be a blood pressure monitor that stores measured blood pressure values in a memory in association with measurement time information and measurement conditions, calculates average values obtained by averaging blood pressure values measured multiple times in a specific time slot such as a morning time slot or an evening time slot, calculates a risk value based on the calculation results, and displays the risk value. Accordingly, health guidance for the measurement subject is supported based on biological information such as the blood pressure values.

### SUMMARY OF THE INVENTION

Incidentally, in recent years, attention has been given to the importance of measuring night-time blood pressure in treatment of hypertension. It is known that examples of types of night-time hypertension (broad definition) include a night-time hypertension type (narrow definition) in which hypertension occurs at night but not in the early morning, an early-morning hypertension (morning surge) type in which hypertension does not occur at night but does occur in the early morning, and a night-time/early-morning hypertension type in which hypertension occurs at night and in the early morning. Because these types are different from each other, the causes of the symptoms differ, and therefore treatment methods also differ. Accordingly, it is convenient to be able to intuitively understand which type the patient belongs to using a display screen of a hospital terminal operated by a doctor, for example.

However, as far as the inventor of the present invention knows, there has not conventionally been an apparatus that performs display such that a user (examples of which include medical professionals such as doctors and nurses; the same follows below) can intuitively understand which type a patient (measurement subject) belongs to regarding night-time hypertension (broad definition).

In view of this, the present invention aims to provide a blood pressure related information display apparatus that displays information related to blood pressure of a measurement subject on a display screen and can perform display such that a user can intuitively understand which type a measurement subject belongs to regarding night-time hypertension (broad definition).

Also, the present invention aims to provide a program for causing a computer to execute a method for displaying information related to blood pressure of a measurement subject on a display screen, according to which it is possible to perform display such that a user can intuitively understand which type a measurement subject belongs to regarding night-time hypertension (broad definition).

In order to solve the foregoing problems, the blood pressure related information display apparatus of the present invention is a blood pressure related information display apparatus configured to display information related to blood pressure of a measurement subject on a display screen, including: a blood pressure data acquisition unit configured to acquire blood pressure data including at least early-morning blood pressure data measured in the morning and night-time blood pressure data measured at night in a certain period for the measurement subject; and a display control unit configured to set one of early-morning blood pressure and night-time blood pressure on a horizontal axis and the other on a vertical axis, and perform graph display of a point determined by the early-morning blood pressure data and the night-time blood pressure data on the display screen.

The “period” described above indicates a period serving as a unit for understanding a blood pressure state of the measurement subject. For example, “period” indicates a period starting from a most-recent time of examination of the measurement subject at a hospital to a current time of examination.

With the blood pressure related information display apparatus of the present invention, the blood pressure data acquisition unit acquires blood pressure data including at least early-morning blood pressure data measured in the morning and night-time blood pressure data measured at night in a certain period for the measurement subject. The display control unit sets one of early-morning blood pressure and night-time blood pressure on a horizontal axis and the other on the vertical axis and performs graph display of a point determined by the early-morning blood pressure data and the night-time blood pressure data on the display screen. Accordingly, by viewing the point subjected to graph display on the display screen, the user can intuitively understand which type the measurement subject belongs to regarding night-time hypertension (broad definition), or in other words, whether the measurement subject belongs to a night-time hypertension type (narrow definition), an early-morning hypertension (morning surge) type, or a night-time/early-morning hypertension type. Accordingly, the user can quickly make a treatment plan corresponding to the type of the measurement subject.

With a blood pressure related information display apparatus of a preferred embodiment, the display control unit performs graph display of a first reference line indicating a reference value for determining early-morning hypertension and a second reference line indicating a reference value for determining night-time hypertension on the display screen, and performs display of which type the point on the display screen is to be classified into among a night-time hypertension type in which hypertension occurs at night but not in the early morning, an early-morning hypertension type in which hypertension does not occur at night but does occur in the early morning, a night-time/early-morning hypertension type in which hypertension occurs at night and in the early morning, and a normal type in which hypertension does not occur at night or in the early morning.

With the blood pressure related information display apparatus of the preferred embodiment, the region determined by the horizontal axis and the vertical axis is divided into four partial regions corresponding to the night-time hypertension type (narrow definition), the early-morning hypertension (morning surge) type, the night-time/early-morning hypertension type, and the normal type by the first reference line and the second reference line. Also, the point determined by the early-morning blood pressure data and the night-time blood pressure data falls within one of the four partial regions. Accordingly, the user can furthermore intuitively and accurately understand which type the measurement subject belongs to regarding night-time hypertension (broad definition).

With a blood pressure related information display apparatus of a preferred embodiment, the blood pressure data acquisition unit acquires blood pressure data including the early-morning blood pressure data and the night-time blood pressure data, which were measured a plurality of times in the period, and includes an arithmetic unit configured to carry out statistical processing respectively on the early-morning blood pressure data and the night-time blood pressure data of the measurement subject to obtain an early-morning blood pressure data average value and a night-time blood pressure data average value, which indicate average values of the period, and the display control unit performs control for performing graph display of a point determined by the early-morning blood pressure data average value and the night-time blood pressure data average value with one of early-morning blood pressure and night-time blood pressure on a horizontal axis and the other on a vertical axis.

With the blood pressure related information display apparatus of the preferred embodiment, the blood pressure data acquisition unit acquires blood pressure data including the early-morning blood pressure data and the night-time blood pressure data, which were measured a plurality of times in the period. The arithmetic unit carries out statistical processing respectively on the early-morning blood pressure data and the night-time blood pressure data of the measurement subject and obtains an early-morning blood pressure data average value and a night-time blood pressure data average value, which indicate average values of the period. The display control unit performs control for performing graph display of a point determined by the early-morning blood pressure data average value and the night-time blood pressure data average value with one of early-morning blood pressure and night-time blood pressure on a horizontal axis and the other on a vertical axis on the display screen. With the blood pressure related information display apparatus, the point on the display screen indicates the early-morning blood pressure data average value and the night-time blood pressure data average value of the period. Accordingly, the reliability of the display content increases.

With a blood pressure related information display apparatus of a preferred embodiment, the blood pressure data acquisition unit sequentially acquires blood pressure data including the early-morning blood pressure data and the night-time blood pressure data measured a plurality of times for a plurality of different periods, the arithmetic unit carries out statistical processing respectively on the early-morning blood pressure data and the night-time blood pressure data of each period to obtain early-morning blood pressure data average values and night-time blood pressure data average values, which indicate average values of the respective periods, and the display control unit performs control for subjecting points determined by the early-morning blood pressure data average values and the night-time blood pressure data average values of the respective periods to graph display simultaneously and performing display with reference signs indicating a period order attached to the points of the periods on the display screen.

A “reference sign” may be a number, a letter, or a symbol.

With the blood pressure related information display apparatus of the preferred embodiment, the blood pressure data acquisition unit sequentially acquires blood pressure data including the early-morning blood pressure data and the night-time blood pressure data measured a plurality of times for a plurality of different periods. The arithmetic unit carries out statistical processing respectively on the early-morning blood pressure data and the night-time blood pressure data of each period to obtain early-morning blood pressure data average values and night-time blood pressure data average values, which indicate average values of the respective periods. The display control unit performs control for subjecting points determined by the early-morning blood pressure data average values and the night-time blood pressure data average values of the respective periods to graph display simultaneously and performing display with reference signs indicating a period order attached to the points of the periods on the display screen. With the blood pressure related information display apparatus, the points determined using the early-morning blood pressure data average values and the night-time blood pressure data average values of the respective periods are simultaneously subjected to graph display on the display screen. Moreover, display is performed with reference signs indicating the period order attached to the points of the respective periods. Accordingly, the user can intuitively understand the transitions in the blood pressure state of the measurement subject in each period. Accordingly, the user can quickly evaluate the effect of the past treatment according to the transitions in the blood pressure state (symptoms) of the measurement subject in each period, and it is easy to suitably make a future treatment plan based on the evaluation.

With a blood pressure related information display apparatus of a preferred embodiment, the display control unit sets the early-morning blood pressure on a horizontal axis and the night-time blood pressure on a vertical axis.

With the blood pressure related information display apparatus of the preferred embodiment, the early-morning blood pressure is set on the horizontal axis and the night-time blood pressure is set on the vertical axis. Due to the fact that the vertical axis tends to be emphasized in common graphs, the night-time blood pressure can be emphasized with the blood pressure related information display apparatus.

The program of the present invention is a program for causing a computer to execute a method for displaying information related to blood pressure of a measurement subject on a display screen, the method including: a first step of acquiring blood pressure data including at least early-morning blood pressure data measured in the morning and night-time blood pressure data measured at night in a certain period for the measurement subject; and a second step of setting one of early-morning blood pressure and night-time blood pressure on a horizontal axis and the other on a vertical axis, and performing graph display of a point determined by the early-morning blood pressure data and the night-time blood pressure data on the display screen.

With the program of the present invention, it is possible to cause a computer to execute a method for displaying information related to blood pressure of a measurement subject on a display screen, as follows. That is, in the first step, blood pressure data including at least the early-morning blood pressure data measured in the morning and the night-time blood pressure data measured at night is acquired in a certain period for the measurement subject. In the second step, one of early-morning blood pressure and night-time blood pressure is set on a horizontal axis and the other is set on a vertical axis, and a point determined by the early-morning blood pressure data and the night-time blood pressure data is subjected to graph display on the display screen. Accordingly, by viewing the point subjected to graph display on the display screen, the user can intuitively understand which type the measurement subject belongs to regarding night-time hypertension (broad definition), or in other words, whether the measurement subject belongs to a night-time hypertension type (narrow definition), an early-morning hypertension (morning surge) type, or a night-time/early-morning hypertension type.

Accordingly, the user can quickly make a treatment plan corresponding to the type of the measurement subject.

As is evident from the description above, with the blood pressure related information display apparatus and program of the present invention, it is possible to perform display such that the user can intuitively understand which hypertension type the measurement subject belongs to.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a block diagram showing a preferred embodiment in which a blood pressure related information display apparatus of the present invention is configured as a system on a network.

FIG. 2 is a block diagram showing a configuration of a hospital terminal included in the system.

FIG. 3 is a block diagram showing a configuration of a server included in the system.

FIG. 4 is a block diagram showing a configuration of a blood pressure monitor included in the system.

FIG. 5 is a diagram showing an overall operation flow of the server.

FIG. 6 is a diagram showing an overall operation flow of the hospital terminal.

FIG. 7 is a diagram illustrating an exemplary image displayed on a display screen of the hospital terminal in the case where an item “blood pressure value transition (90-day display)” in a blood pressure evaluation item list is selected.

FIG. 8 is a diagram illustrating an exemplary image displayed on a display screen of a display unit of the hospital terminal in the case where an item “period average value (most-recent/current comparison)” in the blood pressure evaluation item list is selected.

FIG. 9 is a diagram illustrating an exemplary image displayed on the display screen of the display unit of the hospital terminal in the case where an item “type of night-time hypertension” in the blood pressure evaluation item list is selected.

FIG. 10 is a diagram illustrating another exemplary image displayed on the display screen of the display unit of the hospital terminal in the case where the item “type of night-time hypertension” in the blood pressure evaluation item list is selected.

FIG. 11 is a diagram illustrating yet another exemplary image displayed on a display screen of a display unit of the hospital terminal in the case where the item “type of night-time hypertension” in the blood pressure evaluation item list is selected.

FIG. 12 is a diagram illustrating yet another exemplary image displayed on the display screen of the display unit of the hospital terminal in the case where the item “type of night-time hypertension” in the blood pressure evaluation item list is selected.

FIG. 13 is a diagram showing an overall operation flow of the hospital terminal in a modified example in which the function of the server is incorporated in the hospital terminal.

### DETAILED DESCRIPTION OF THE PREFERRED EMBODIMENTS

Hereinafter, preferred embodiments of the present invention will be described in detail with reference to the drawings.

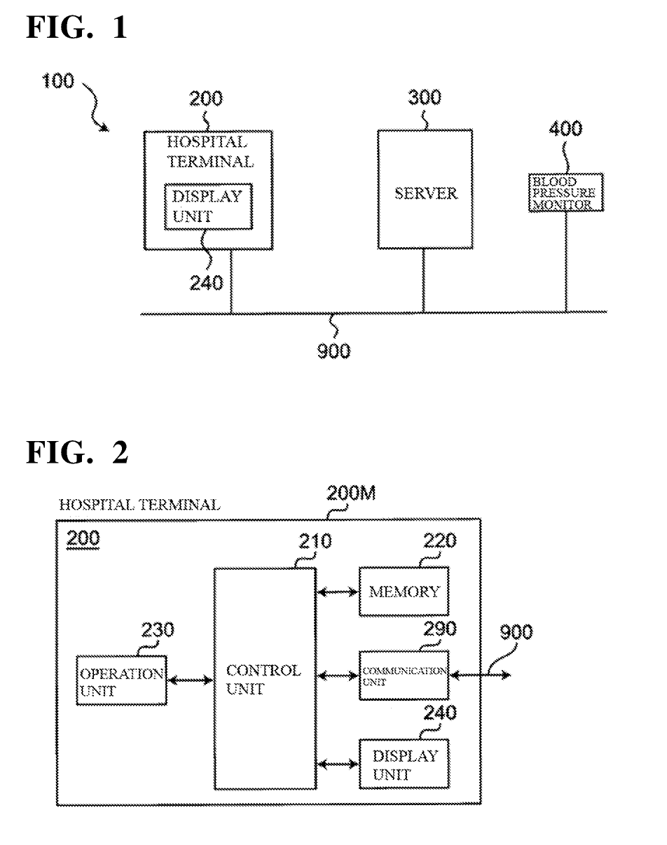

FIG. 1 shows an example in which the blood pressure related information display apparatus of the present invention is configured as a system (indicated overall by reference numeral 100) according to a preferred embodiment on a network. The system 100 includes a hospital terminal 200 having a display unit 240 that serves as a display screen, a server 300, and a blood pressure monitor 400. The hospital terminal 200, the server 300, and the blood pressure monitor 400 can communicate with each other via the network 900.

As shown in FIG. 2, the hospital terminal 200 includes a main body 200M, and a control unit 210, a memory 220, an operation unit 230, the display unit 240 serving as the display screen, and a communication unit 290, which are mounted in the main body 200M. The hospital terminal 200 is composed of a commercially-available laptop personal computer, has application software (computer programs) installed so as to execute later-described processing, and can access the server 300.

The control unit 210 includes a CPU (Central Processing Unit) and auxiliary circuits thereof, controls the units of the hospital terminal 200, and executes later-described processing in accordance with programs and data stored in the memory 220. That is, data input from the operation unit 230 and the communication unit 290 is processed, and the processed data is stored in the memory 220, displayed on the display unit 240, and output from the communication unit 290.

The memory 220 includes a RAM (Random Access Memory) used as a work region needed for executing a program using the control unit 210, and a ROM (Read Only Memory) for storing basic programs to be executed by the control unit 210. Also, a semiconductor memory (memory card, SSD (Solid State Drive)) or the like may be used as a storage medium of an auxiliary storage apparatus for assisting the storage region of the memory 220.

In this example, the operation unit 230 is constituted by a keyboard and mouse, and inputs an operation signal indicating an operation performed by a user (assumed to be a doctor in this example) to the control unit 210. Also, the operation unit 230 may be constituted by another operation device such as a touch panel instead of or in addition to the keyboard and mouse.

The display unit 240 includes a display screen (e.g., LCD (Liquid Crystal Display), EL (Electroluminescence) display, or the like). The display unit 240 is controlled by the control unit 210 to display a predetermined video image on the display screen.

The communication unit 290 transmits information from the control unit 210 to the server 300 via the network 900.

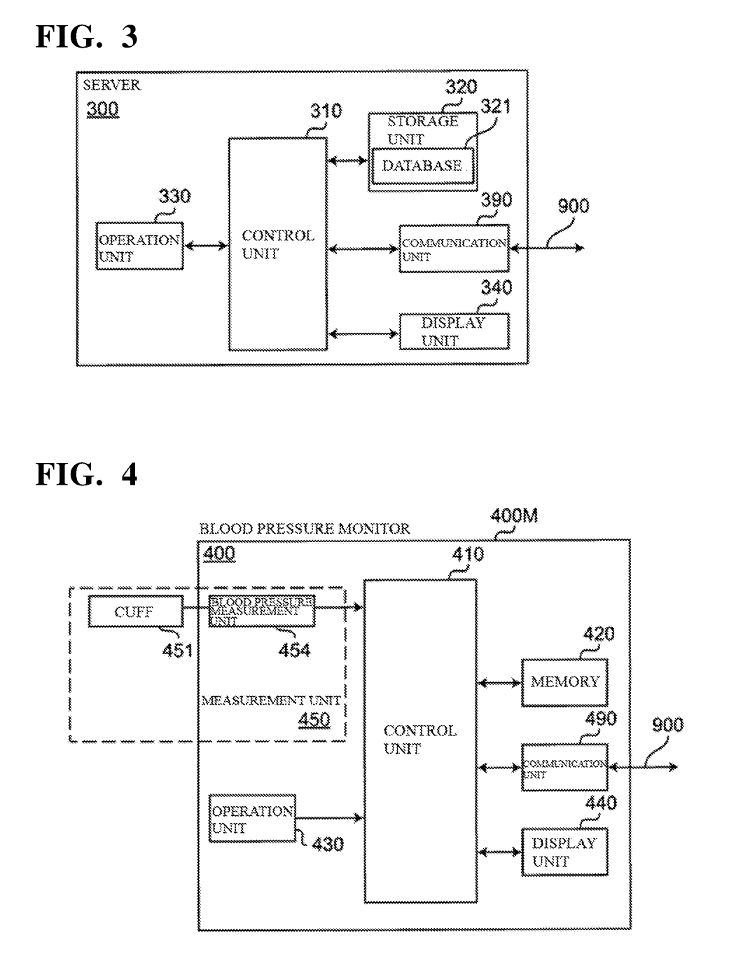

As shown in FIG. 3, the server 300 includes a control unit 310, a storage unit 320, an operation unit 330, a display unit 340, and a network communication unit 390. The server 300 is a general-purpose computer apparatus on which a program (software) is installed in order to perform the later-described processing.

The control unit 310 includes a CPU and peripheral circuits thereof, controls the units of the server 300, executes predetermined processing in accordance with programs and data stored in the storage unit 320, processes data input from the operation unit 330 and the communication unit 390, stores the processed data in the storage unit 320, displays the processed data on the display unit 340, and causes the processed data to be output from the communication unit 390.

The storage unit 320 includes a RAM used as a work region needed for executing a program using the control unit 310 and a ROM for storing basic programs to be executed by the control unit 310. The storage unit 320 is provided with a database 321 including blood pressure measurement data sent from many measurement subjects. Also, a magnetic disk (HD (Hard Disk), FD (Flexible Disk)), an optical disk (CD (Compact Disc), DVD (Digital Versatile Disk), BD (Blu-ray Disc)), a magneto-optical disk (MO), a semiconductor memory (memory card, SSD), or the like may be used as the storage medium of the auxiliary storage apparatus for assisting the storage region of the storage unit 320.

In this example, the operation unit 330 is constituted by a keyboard and mouse, and inputs an operation signal indicating an operation performed by a user to the control unit 310. Also, the operation unit 330 may be constituted by another operation device such as a touch panel instead of or in addition to the keyboard and mouse.

The display unit 340 includes a display screen (e.g., LCD, EL display, or the like). The display unit 340 is controlled by the control unit 310 to display a predetermined video image on a display.

The network communication unit 390 transmits information from the control unit 310 to another apparatus (the hospital terminal 200 in this example) via the network 900, receives information transmitted from the other apparatus via the network 900, and transfers the information to the control unit 310.

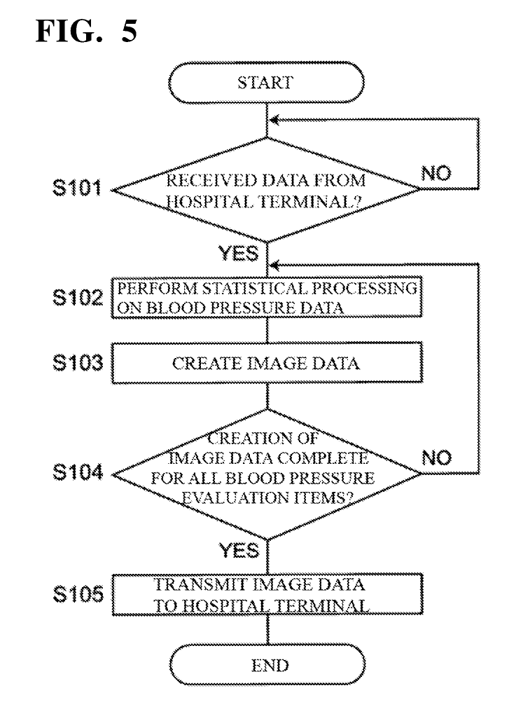

As shown in FIG. 4, the blood pressure monitor 400 includes a casing 400M, and a control unit 410, a memory 420, an operation unit 430, a display unit 440, a measurement unit 450 serving as a blood pressure data acquisition unit, and a communication unit 490, which are mounted in the casing 400M.

In response to an operator performing an operation on an operation device (not shown) such as an operation button or operation key, the operation unit 430 inputs an operation signal corresponding to the content of the operation to the control unit 410.

The measurement unit 450 includes a cuff 451 and a blood pressure measurement unit 454 for measuring blood pressure, and acquires measured values of a systolic blood pressure (SBP), a diastolic blood pressure (DBP), and a pulse rate from a patient.

The memory 420 stores data of programs for controlling the blood pressure monitor 400, setting data for setting various functions of the blood pressure monitor 400, data of the measurement results, and the like. Also, the memory 420 is used as a work memory or the like for when a program is executed.

The control unit 410 includes a CPU and controls the memory 420, the display unit 440, and the communication unit 490 in accordance with a program for controlling the blood pressure monitor 400 stored in the memory 420, in response to operation signals from the operation unit 430, and based on a detection signal from the measurement unit 450. The control unit 410 can execute a mode of automatically measuring blood pressure at night in addition to a mode of measuring blood pressure when a “measurement” instruction is input using the operation unit 430.

The display unit 440 includes a display, an indicator, and the like and displays predetermined information in accordance with a control signal from the control unit 410.

The communication unit 490 is controlled by the control unit 410 to transmit the predetermined information to an external apparatus via the network 900, receive information from the external apparatus via the network 900, and transfer the information to the control unit 410. Communication via the network 900 may be wireless or wired. In this preferred embodiment, the network 900 is the Internet, but there is no limitation to this, and the network 900 may be another type of network such as an intra-hospital LAN (Local Area Network), or the network 900 may perform one-on-one communication using a USB cable or the like.

The system 100 is used as follows.

i) The measurement subject uses the blood pressure monitor 400 to automatically measure the measurement subject's blood pressure at home, for example, in the morning and evening each day, and at night at least twice a week. When the measurement ends, the blood pressure monitor 400 transmits the measured blood pressure data along with the measurement date/time thereof to the server 300 via the network 900. Accordingly, blood pressure data including early-morning blood pressure data measured in the morning, evening blood pressure data measured in the evening, and night-time blood pressure data measured at night is transmitted to the server 300.

Here, “blood pressure data” broadly indicates the systolic blood pressure (SBP), the diastolic blood pressure (DBP), and the pulse rate.

In this example, the daily blood pressure measurement performed by the measurement subject is performed sequentially over a period starting from the third most-recent examination before the current examination at the hospital to the second most-recent examination (the second most-recent examination period), over a period starting from the second most-recent examination to the most-recent examination (the most-recent examination period), and over a period starting from the most-recent examination to the current examination (current examination period).

ii) The server 300 functions as a blood pressure data acquisition unit and receives the blood pressure data from the blood pressure monitor 400 while the server 300 is operating. Also, the received blood pressure data is stored along with the measurement time/date thereof in the storage unit 320.

iii) When the doctor performs an operation on the operation unit 230 of the hospital terminal 200 while the hospital terminal 200 is operating and selects “blood pressure evaluation display” relating to the measurement subject (patient), the control unit 210 of the hospital terminal 200 transmits date data indicating the current examination date along with a request to acquire the blood pressure evaluation result to the server 300.

iv) As shown in step S101 in FIG. 5, the control unit 310 of the server 300 waits for the data including the date data to be sent from the hospital terminal 200 via the network 900 while the server 300 is operating. Upon receiving the data from the hospital terminal 200 via the communication unit 390 from the network 900 (YES in step S101), the control unit 310 functions as an arithmetic unit to perform statistical processing on the stored blood pressure data for a pre-determined blood pressure evaluation item (step S102).

Examples of the pre-determined blood pressure evaluation item include “blood pressure value transition (90-day display)”, “period average value (most-recent/current comparison)”, and “type of night-time hypertension”.

Specifically, in the case of performing statistical processing regarding “blood pressure value transition”, the control unit 310 calculates representative values for the systolic blood pressure (SBP) and representative values for the diastolic blood pressure (DBP) for morning, evening, and night of each day over a 90-day period prior to the current examination date, for example. In this example, when the measurement subject performs multiple (e.g., two) instances of measurement in each of morning, evening, and night, the representative values are the respective average values of the systolic blood pressures (SBP) and the diastolic blood pressures (DBP) of those multiple instances.

Also, in the case of performing statistical processing for the “period average value”, according to the number of measurement days, the control unit 310 calculates averages of the blood pressure values (indicates representative values for the systolic blood pressures (SBP) and representative values for the diastolic blood pressures (DBP); the same follows below) for the morning, evening, and night for each day of the measurement subject measured in a period starting from the most-recent examination to the current examination (the current examination period) as the period average values of the current examination period. That is, for the current examination period, a total of six period average values are calculated, which include the period average value for the systolic blood pressures (SBP) measured in the morning, the period average value for the diastolic blood pressures (DBP) measured in the morning, the period average value for the systolic blood pressures (SBP) measured in the evening, the period average value for the diastolic blood pressures (DBP) measured in the evening, the period average value for the systolic blood pressures (SBP) measured at night, and the period average value for the diastolic blood pressures (DBP) measured at night.

Similarly, according to the number of measurement days, the control unit 310 calculates averages of the blood pressure values for the morning, evening, and night of each day in the period starting from the second most-recent examination to the most-recent examination (most-recent examination period) as the period average values for the most-recent examination period. That is, a total of six period average values are calculated for the most-recent examination period, similarly to those in the current examination period. Also, according to the number of measurement days, averages of the blood pressure values for the morning, evening, and night of each day in the period starting from the third most-recent examination prior to the current examination to the second most-recent examination (the second most-recent examination period) are calculated as the period average values for the second most-recent examination period. That is, a total of six period average values are calculated for the second most-recent examination period. Furthermore, the averages of blood pressure values for the morning, evening, and night for each day in the period starting from the time when measurement was first performed by the blood pressure monitor 400 to the first examination (first examination period) are calculated as period average values for the first examination period. That is, a total of six period average values are calculated for the first examination period.

In order to evaluate the “type of night-time hypertension”, a value obtained using statistical processing on the “period average values” is used (details of which will be described later).

Next, the control unit 310 creates image data (the image data will be described later) to be displayed on the display unit 240 of the hospital terminal 200 for the blood pressure evaluation items subjected to statistical processing (step S103 in FIG. 5).

Next, when the creation of the image data for all of the blood pressure evaluation items is complete (YES in step S104), the control unit 310 stores the created image data in the database 321. In addition, the created image data is transmitted from the communication unit 390 to the hospital terminal 200 via the network 900 (step S105).

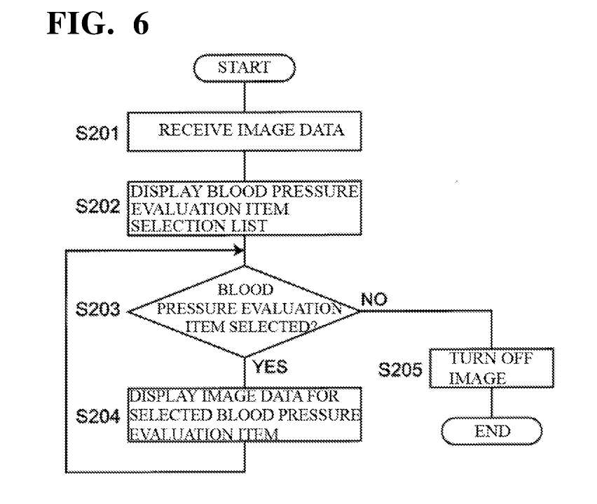

v) As shown in the flowchart in FIG. 6, the hospital terminal 200 receives the image data from the network 900 via the communication unit 290 (step S201) and causes the memory 220 to function as the image data storage unit to store the image data. Next, the control unit 210 of the hospital terminal 200 displays a blood pressure evaluation item list (illustrated in Table 1 below) for prompting the doctor to select a blood pressure evaluation item on the display screen of the display unit 240 (step S202).

TABLE 1

Blood pressure evaluation item
Blood pressure transition (90-day display)
Blood pressure transition (60-day display)
Blood pressure transition (30-day display)
Period average value (most-recent/current comparison)
Period average value (current)
Type of night-time hypertension
. . .

Here, it is assumed that the doctor selects (e.g., clicks on with the mouse) an item in the blood pressure evaluation item list (YES in step S203 in FIG. 6). Upon doing so, the control unit 210 functions as the display control unit to read out image data (to be described later) related to the blood pressure evaluation result of the selected item from the memory 220 and display the blood pressure evaluation result on the display screen of the display unit 240 (step S204 in FIG. 6). On the other hand, when an operation has not been performed for a certain amount of time (e.g., 60 seconds) in the state in which the blood pressure evaluation item list (Table 1) is displayed on the display screen of the display unit 240 (NO in step S203 of FIG. 6), the control unit 210 turns off the image to save power (step S205 in FIG. 6).

### (a) Blood Pressure Value Transition (90-Day Display)

The item “blood pressure value transition (90-day display)” in the blood pressure evaluation item list of Table 1 above means performing graph display of transitions in the systolic blood pressure values (SBP) and diastolic blood pressure values (DBP) (indicates the already-mentioned representative values; the same follows below) measured in the morning, evening, and night over a 90-day period prior to the current examination date.

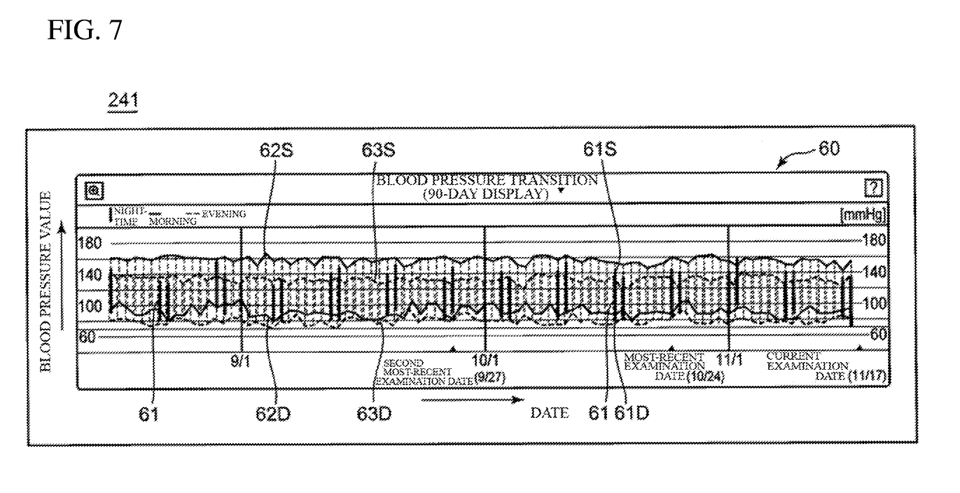

If the item “blood pressure value transition (90-day display)” is selected, an image 60 shown in FIG. 7 for example is displayed on the display screen 241 of the display unit 240 of the hospital terminal 200.

With this image 60, the date is indicated in the horizontal direction, and the blood pressure value (in units of mmHg) is indicated in the vertical direction, and the blood pressure values of the measurement subject for morning and evening of each day corresponding to an approximate 90-day period prior to the current examination date (November 17) in this example) are displayed as solid line graphs 62S and 62D and dotted line graphs 63S and 63D. The line graph 62S indicates SBP measured in the morning. The line graph 62D indicates DBP measured in the morning. The line graph 63S indicates SBP measured in the evening. The line graph 63D indicates DBP measured in the evening.

Also, with the image 60, the blood pressure values of the measurement subject measured at night twice a week are indicated as bar graphs 61, 61, and the like. The upper ends 61S of the bar graphs 61 indicate SBP. The lower ends 61D of the bar graphs 61 indicate DBP.

These line graphs 62S, 62D, 63S, and 63D, and bar graphs 61, 61, . . . are created using the processing described above (in particular, steps S102 and S103 in FIG. 5).

By viewing the image 60, the doctor can intuitively understand the temporal transitions in the blood pressure values of the measurement subject in the approximate 90-day period prior to the current examination date (November 17).

The image 60 also displays that the most-recent examination date for the measurement subject was October 24 and the second most-recent examination date was September 27.

Note that when the items “blood pressure value transition (60-day display)” or “blood pressure value transition (30-day display)” in Table 1 are selected, 60-day display or 30-day display, which correspond to display with a reduced number of days in the horizontal direction in FIG. 7, is performed instead of 90-day display.

### (b) Period Average Value (Most-Recent/Current Comparison)

The item “period average value (most-recent/current comparison)” in the blood pressure evaluation item list in Table above means comparing the period average value of the blood pressure values measured in the evening, night, and morning for the current evaluation period and the period average value of the blood pressure values measured in the evening, night, and morning for the most-recent evaluation period and performing graph display.

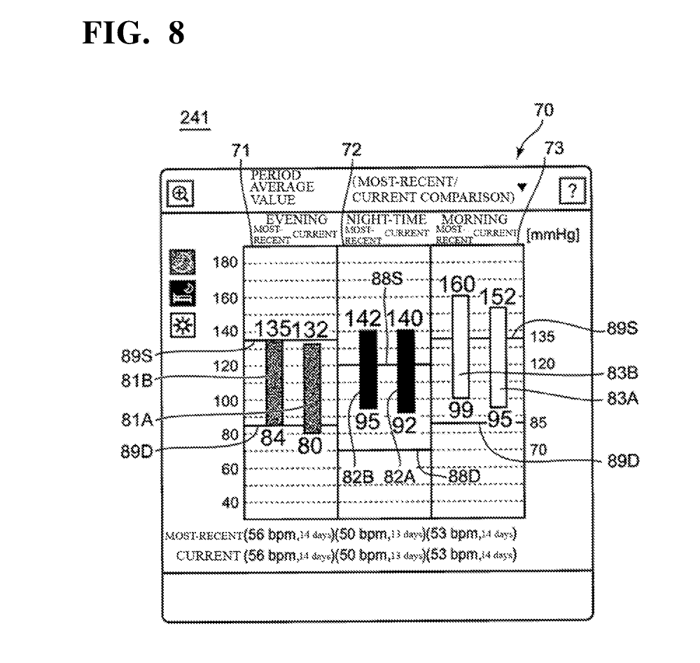

If the item “period average value (most-recent/current comparison)” is selected, an image 70 shown in FIG. 8 for example is displayed on the display screen 241 of the display unit 240 of the hospital terminal 200.

In the image 70, an evening field 71, a night field 72, and a morning field 73 are aligned in the horizontal direction, blood pressure values (in units of mmHg) are aligned in the vertical direction, and the period average values of the blood pressure values for the most-recent and current examination periods are displayed in alignment in bar graphs 81B and 81A, 82B and 82A, and 83B and 83A in the fields 71, 72, and 73.

Specifically, in the evening field 71, the period average values of the blood pressure values measured in the evening for the most-recent examination period are displayed using the bar graph 81B (in this example, the period average value for SBP was 135 mmHg and the period average value for DBP was 84 mmHg). Also, the period average values for the blood pressure values measured in the evening for the current examination period are displayed using the bar graph 81A (in this example, the period average value for SBP was 132 mmHg and the period average value for DBP was 80 mmHg). In the night field 72, the period average values for the blood pressure values measured at night for the most-recent examination period are displayed using the bar graph 82B (in this example, the period average value for SBP was 142 mmHg and the period average value for DBP was 95 mmHg). Also, the period average values for the blood pressure values measured at night for the current examination period are displayed using the bar graph 82A (in this example, the period average value for SBP was 140 mmHg and the period average value for DBP was 92 mmHg). In the morning field 73, the period average values of the blood pressure values measured in the morning for the most-recent examination period are displayed using the bar graph 83B (in this example, the period average value for SBP was 160 mmHg and the period average value for DBP was 99 mmHg). Also, the period average values for the blood pressure values measured in the morning for the current examination period are displayed using the bar graph 83A (in this example, the period average value for SBP was 152 mmHg and the period average value for DBP was 95 mmHg).

These bar graphs 81B and 81A, 82B and 82A, and 83B and 83A are created using the above-described processing (particularly steps S102 and S103 in FIG. 5).

By viewing the image 70, the doctor can intuitively understand how the period average values of the blood pressure values for evening, night, and morning of the above-described measurement subject have changed in the period between the most-recent examination period and the current examination period.

Also, in the evening field 71 and the morning field 73, a boundary line 89S indicating a reference value 135 mmHg for determining whether or not the SBP is a high blood pressure, and a boundary line 89D indicating a reference value 85 mmHg for determining whether or not the DBP is a high blood pressure are displayed. A boundary line 88S indicating the reference value 120 mmHg for whether or not the SBP is a high blood pressure, and a boundary line 88D indicating a reference value 70 mmHg for whether or not the DBP is a high blood pressure are displayed in the night field 72. Accordingly, the doctor can intuitively and accurately understand whether or not the period average values of the blood pressure values for the evening, night, and morning of the measurement subject are high blood pressures in the most-recent examination period and the current examination period.

Also, the period average values of the pulse rates and the number of measurement dates for the most-recent and current examination periods are displayed as numerical values below the evening field 71, the night field 72, and the morning field 73 of the image 70. For example, the period average values of the pulse rates measured in the evening for the most-recent and current examination periods are both 56 bpm. Also, the most-recent and current examination periods each had 14 measurement days. Accordingly, the doctor can check whether or not the most-recent and current period average values are trustworthy (check the degree of reliability).

Note that when the item “period average value (current)” in Table 1 is selected, graph display of only the period average values of the blood pressure values for the evening, night, and morning for the current examination period is performed instead of most-recent/current comparison.

### (c) Type of Night-Time Hypertension

The item “type of night-time hypertension” in the blood pressure evaluation item list in Table 1 above means performing graph display of which type the measurement subject is to be classified into among a normal type in which neither night-time hypertension nor early-morning hypertension occur, a night-time hypertension type (narrow definition) in which night-time hypertension occurs but early-morning hypertension does not, an early-morning hypertension type in which night-time hypertension does not occur but early-morning hypertension does, and a night-time/early-morning hypertension type in which both night-time hypertension and early-morning hypertension occur.

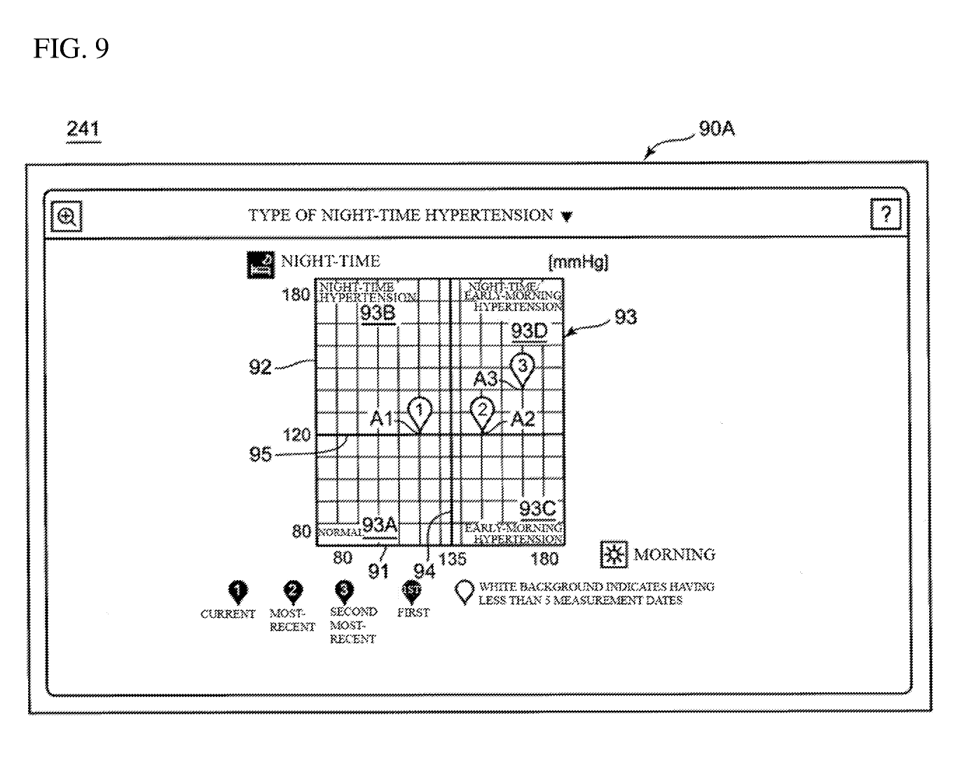

If the item “type of night-time hypertension” is selected, an image 90A shown in FIG. 9 for example is displayed on the display screen 241 of the display unit 240 of the hospital terminal 200.

In the image 90A, early-morning blood pressure is set on the horizontal axis 91 and night-time blood pressure is set on the vertical axis 92. Night-time blood pressure is set on the vertical axis 92 in order to emphasize the night-time blood pressure. The unit of the horizontal axis 91 and the vertical axis 92 is mmHg. Points A1, A2, and A3, which are determined by the early-morning blood pressure data measured in the morning and the night-time blood pressure data measured at night, are subjected to graph display in a graph region 93 determined by the horizontal axis 91 and the vertical axis 92 for a certain measurement subject (denoted as “A”).

In this example, the point A1 is a point that is determined using the period average value of the systolic blood pressures (SBP) measured in the morning and the period average value of the systolic blood pressures (SBP) measured at night for the current examination period. Also, the point A2 is a point that is determined using the period average value of the systolic blood pressures (SBP) measured in the morning and the period average value of the systolic blood pressures (SBP) measured at night for the most-recent examination period. Also, the point A3 is a point that is determined using the period average value of the systolic blood pressures (SBP) measured in the morning and the period average value of the systolic blood pressures (SBP) measured at night for the second most-recent examination period.

The points A1, A2, and A3 are created using the above-described processing (particularly, steps S102 and S103 in FIG. 5).

In this example, the points A1, A2, and A3 of each period are simultaneously subjected to graph display, and numbers 1, 2, and 3, which indicate the period order, are displayed attached to the points A1, A2, and A3 in balloons. A note explaining the meaning of the period order (numbers 1, 2, and 3 indicated by the balloons) is included below the graph region 93. Note that a balloon having a white interior (white background) indicates that the number of measurement days for the period was less than five days. The period order is not limited to being expressed by numbers, and may be expressed using letters, symbols, or other reference signs.

Also, in this example, a first reference line 94 indicating a reference value 135 mmHg for determining early-morning hypertension and a second reference line 95 indicating a reference value 120 mmHg for determining night-time hypertension are subjected to graph display on the display screen 241. Accordingly, the graph region 93 is divided into four partial regions, namely a partial region 93B corresponding to the night-time hypertension type, in which hypertension occurs at night but not in the morning, a partial region 93C corresponding to the early-morning hypertension type, in which hypertension does not occur at night but does occur in the early morning, a partial region 93D corresponding to the night-time/early-morning hypertension type, in which hypertension occurs at night and in the early morning, and a partial region 93A corresponding to a normal type, in which hypertension does not occur at night or in the early morning. The divided regions 93B, 93C, 93D, and 93A of the graph region 93 in which the points A1, A2, and A3 are located indicate the type to which the measurement subject belongs (or belonged; the same follows below) regarding night-time hypertension (broad definition), or indicate that the measurement subject is normal.

Accordingly, by viewing the image 90A, the doctor can intuitively and accurately understand which type the measurement subject A belongs to regarding night-time hypertension (broad definition), or in other words, whether the measurement subject A belongs to the night-time hypertension type (narrow definition), the early-morning hypertension type (morning surge), the night-time/early-morning hypertension type, or the normal type. Accordingly, the doctor can quickly make a treatment plan corresponding to the type of the measurement subject.

Specifically, as indicated by the points A3 and A2, the measurement subject A belonged to the night-time/early-morning hypertension type in the second most-recent examination period and the most-recent examination period. Because the past treatment has been successful in treating the early-morning hypertension, hypertension no longer occurs in the early morning in the current examination period, and as indicated by the point A1, the measurement subject A belongs to the night-time hypertension type (narrow definition). Accordingly, the doctor can easily judge that treatment for night-time hypertension (narrow definition) is to be undertaken as the future treatment plan.

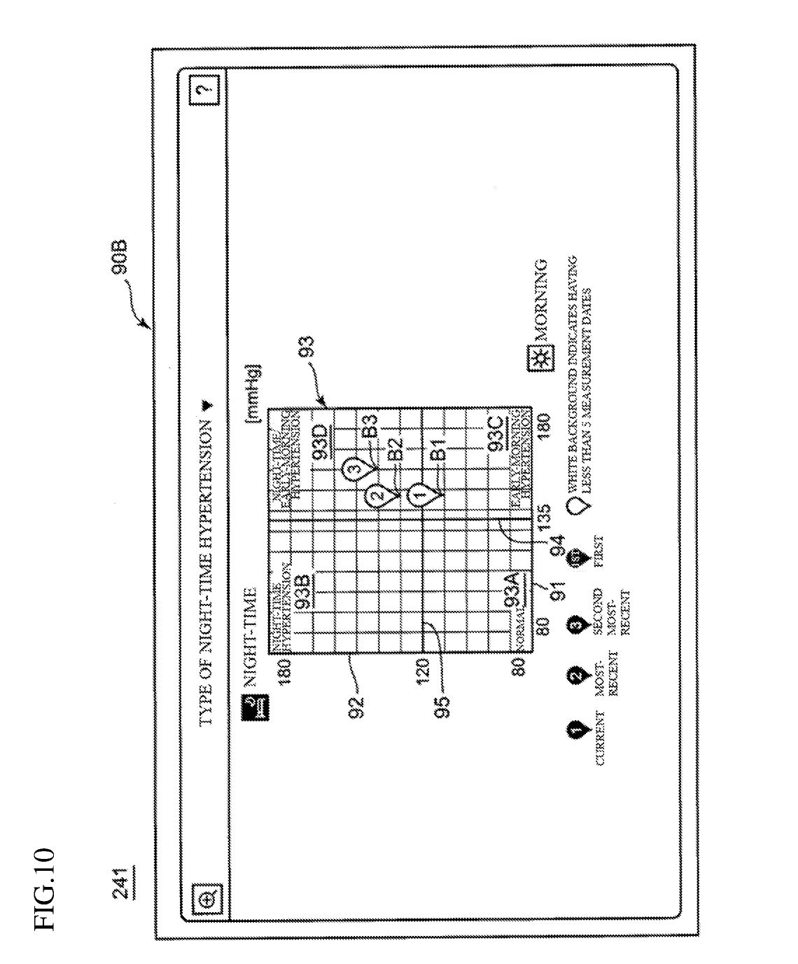

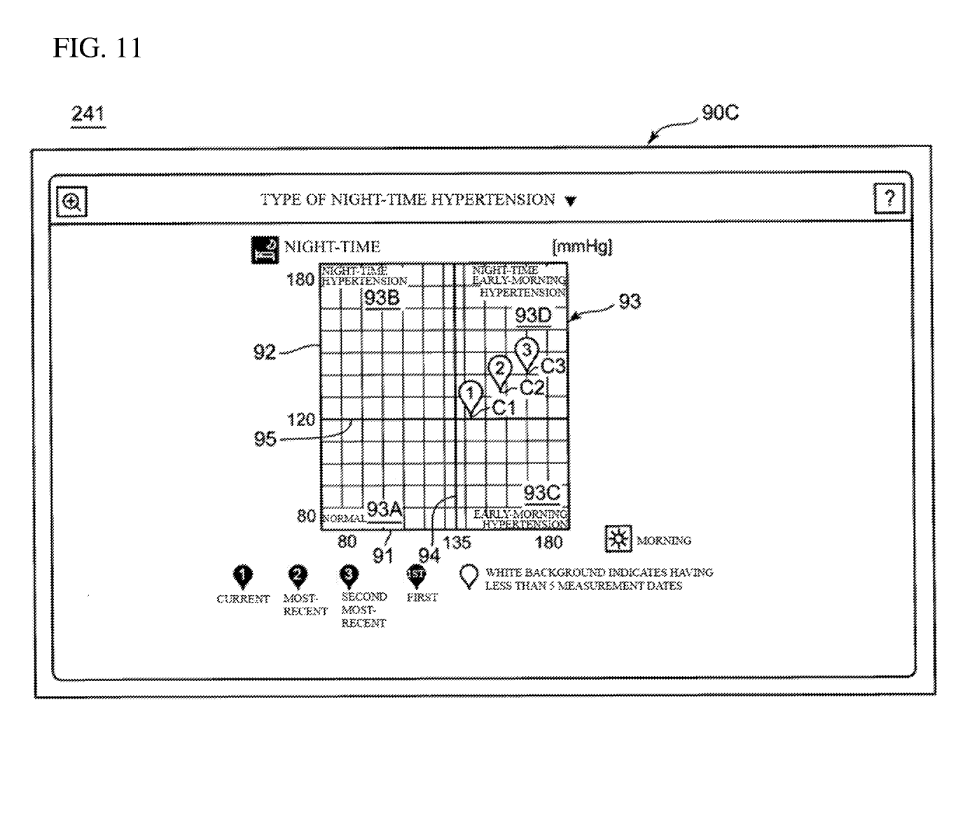

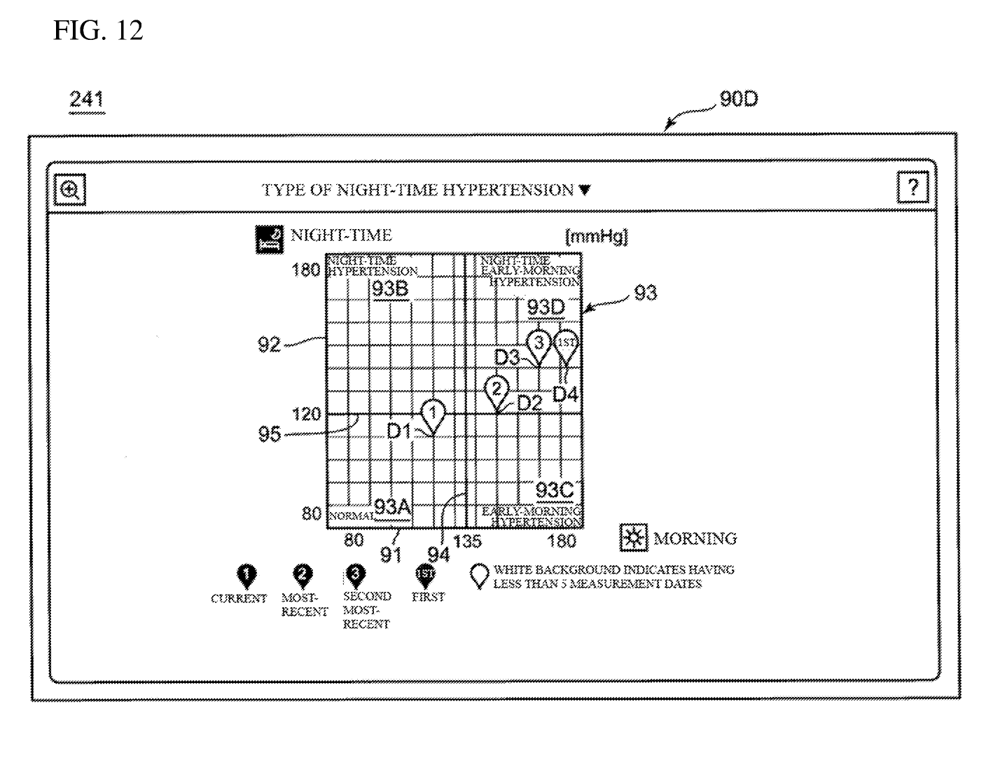

FIG. 10 illustrates an exemplary image 90B displayed on the display screen 241 of the display unit 240 of the hospital terminal 200 in the case where the item “type of night-time hypertension” has been selected for another measurement subject (denoted as “B”). Note that in FIG. 10, elements that are the same as the elements in FIG. 9 are denoted by the same reference signs thereas, and redundant description thereof is not included (the same follows for FIGS. 11 and 12).

As indicated by the points B3 and B2, the measurement subject B belonged to the night-time/early-morning hypertension type in the second most-recent examination period and the most-recent examination period. Because the past treatment was successful in treating night-time hypertension, in the current examination period, hypertension no longer occurs at night, and the measurement subject B belongs to the early-morning hypertension (morning surge) type, as indicated by the point B1. Accordingly, the doctor can easily judge that treatment for early-morning hypertension (morning surge) is to be undertaken as the future treatment plan.

FIG. 11 illustrates an exemplary image 90C displayed on the display screen 241 of the display unit 240 of the hospital terminal 200 in the case where the item “type of night-time hypertension” has been selected for another measurement subject (denoted as “C”).

As indicated by the points C3 and C2, the measurement subject C belonged to the night-time/early-morning hypertension type in the second most-recent examination period and the most-recent examination period. In the current examination period as well, as indicated by the point C1, the measurement subject C still belongs to the night-time/early-morning hypertension type. However, the symptoms (blood pressure state) of the measurement subject C are gradually improving each period and approaching the normal region 93A. Accordingly, the doctor can easily judge that the past treatment is to be continued as the future treatment plan.

FIG. 12 illustrates an exemplary image 90D displayed on the display screen 241 of the display unit 240 of the hospital terminal 200 in the case where the item “type of night-time hypertension” has been selected for another measurement subject (denoted as “D”). Note that the point D4 in FIG. 12 indicates a point determined by the early-morning blood pressure data and the night-time blood pressure data measured in the first examination stage. The point D4 is denoted by a balloon stating “1st” in order to indicate that it is the point of the first examination stage.

As indicated by the points D4, D3, and D2, the measurement subject D belonged to the night-time/early-morning hypertension type in the first examination stage, the second most-recent examination period, and the most-recent examination period. Because the past treatment was successful in treating the night-time/early-morning hypertension, in the current examination period, the measurement subject D belongs to the normal type as indicated by the point D1. Accordingly, the doctor can easily judge whether to continue the past treatment or to switch to a drug with a mild effect, or the like, as the future treatment plan.

Thus, with the images 90A to 90D in FIGS. 9 to 12, the doctor can intuitively and accurately understand which type the measurement subject belongs to regarding night-time hypertension (broad definition). Moreover, because the points displayed in the graph region 93 are denoted by reference signs (numbers) indicating the period order, it is possible to quickly evaluate the effect of the past treatment according to the transitions in the blood pressure state (symptoms) of the measurement subject in each period, and it is easy to suitably establish a future treatment plan based on the evaluation.

Note that it is desirable to display the image 60 shown in FIG. 7, the image 70 shown in FIG. 8, and the image 90A shown in FIG. 9 on the display screen 241 of the display unit 240 of the hospital terminal 200 for a certain measurement subject (e.g., denoted by “A”). Accordingly, the doctor can intuitively understand which type the measurement subject A belongs to regarding night-time hypertension (broad definition) based on the image 90A shown in FIG. 9 and can check blood pressure data (image 60 shown in FIG. 7 and image 70 shown in FIG. 8) to be used as supporting evidence for the determined type.

### Variation 1

In this preferred embodiment, the server 300 created the image data and the display unit 240 of the hospital terminal 200 displayed the image, but there is no limitation to this. Instead of transmitting the image data to the hospital terminal 200, the control unit 310 of the server 300 may transmit only data for image creation and the control unit 210 of the hospital terminal 200 may exclusively create an image.

### Variation 2

Also, in this preferred embodiment, the blood pressure related information display apparatus of the present invention was configured as a system 100 on a network including the hospital terminal 200, the server 300, and the blood pressure monitor 400, but there is no limitation to this.

For example, the blood pressure related information display apparatus of the present invention may be constituted by only the blood pressure monitor 400 and the hospital terminal 200. That is, the control unit 210, the memory 220, the operation unit 230, the display unit 240, and the communication unit 290 of the hospital terminal 200 fulfill the functions of the control unit 310, the storage unit 320, the operation unit 330, the display unit 340, and the network communication unit 390 of the server 300, in addition to their own respective functions. In this case, a program for causing the control unit 210 to execute a method such as the following for displaying information related to the blood pressure of the measurement subject on the display screen is installed in the memory 220 of the hospital terminal 200. Accordingly, the blood pressure related information display apparatus of the present invention can be made small and compact.

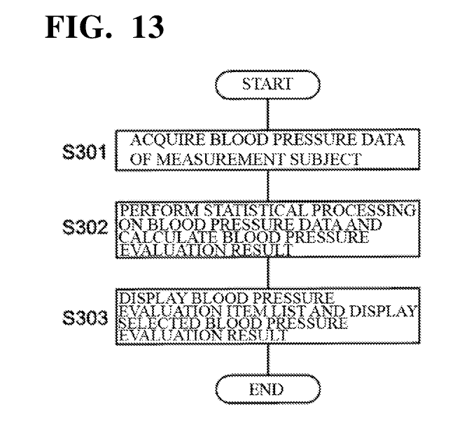

The method includes three main steps as shown in FIG. 13.

In the first step S301 in FIG. 13, blood pressure data of the measurement subject measured by the blood pressure monitor 400 is acquired.

In the second step S302, the pre-determined blood pressure evaluation item for evaluating the state of the blood pressure of the measurement subject is measured each day over a certain period, statistical processing is carried out on the blood pressure data acquired using the above-described first step S301, and thus the blood pressure evaluation result is calculated. The second step S302 corresponds to step S102 in FIG. 5.

In the third step S303, the blood pressure evaluation item list is displayed on the display screen, and the image data relating to the blood pressure evaluation item selected in the blood pressure evaluation item list is displayed. The third step S303 corresponds to steps S202 to S204 in FIG. 6.

The program can be recorded as application software in a recording medium such as a CD, DVD, or flash memory. By installing the application software recorded in the recording medium on a substantial computer apparatus such as a hospital terminal, a personal computer, a PDA (personal digital assistant), or a smartphone, it is possible to cause those computer apparatuses to execute the method for displaying information related to the blood pressure of the measurement subject on a display screen.

### Variation 3

Note that by furthermore incorporating the measurement unit 450 of the blood pressure monitor 400 in such a hospital terminal (in which the function of the server 300 is incorporated), the blood pressure related information display apparatus of the present invention may be constituted by substantially only the hospital terminal.

The above-described preferred embodiments were merely examples and can be modified in various ways without departing from the scope of the present invention. Also, the above-described preferred embodiments can be used separately or in combination. Also, the various features in the different preferred embodiments can be used separately or in combination.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A blood pressure related information display apparatus configured to display information related to blood pressure of a measurement subject on a display screen, comprising:
a blood pressure data acquisition unit configured to acquire blood pressure data including at least early-morning blood pressure data measured in the morning and night-time blood pressure data measured at night in a certain period for the measurement subject; and
a display control unit configured to set one of early-morning blood pressure and night-time blood pressure on a horizontal axis and the other on a vertical axis, and perform graph display of a point determined by the early-morning blood pressure data and the night-time blood pressure data on the display screen.

2. The blood pressure related information display apparatus according to claim 1, wherein
the display control unit performs graph display of a first reference line indicating a reference value for determining early-morning hypertension and a second reference line indicating a reference value for determining night-time hypertension on the display screen, and performs display of which type the point on the display screen is to be classified into among a night-time hypertension type in which hypertension occurs at night but not in the early morning, an early-morning hypertension type in which hypertension does not occur at night but does occur in the early morning, a night-time/early-morning hypertension type in which hypertension occurs at night and in the early morning, and a normal type in which hypertension does not occur at night or in the early morning.

3. The blood pressure related information display apparatus according to claim 1, wherein
the blood pressure data acquisition unit acquires blood pressure data including the early-morning blood pressure data and the night-time blood pressure data, which were measured a plurality of times in the period, and
includes an arithmetic unit configured to carry out statistical processing respectively on the early-morning blood pressure data and the night-time blood pressure data of the measurement subject to obtain an early-morning blood pressure data average value and a night-time blood pressure data average value, which indicate average values of the period, and
the display control unit performs control for performing graph display of a point determined by the early-morning blood pressure data average value and the night-time blood pressure data average value with one of early-morning blood pressure and night-time blood pressure on a horizontal axis and the other on a vertical axis.

4. The blood pressure related information display apparatus according to claim 3, wherein
the blood pressure data acquisition unit sequentially acquires blood pressure data including the early-morning blood pressure data and the night-time blood pressure data measured a plurality of times for a plurality of different periods,
the arithmetic unit carries out statistical processing respectively on the early-morning blood pressure data and the night-time blood pressure data of each period to obtain early-morning blood pressure data average values and night-time blood pressure data average values, which indicate average values of the respective periods, and
the display control unit performs control for subjecting points determined by the early-morning blood pressure data average values and the night-time blood pressure data average values of the respective periods to graph display simultaneously and performing display with reference signs indicating a period order attached to the points of the periods on the display screen.

5. The blood pressure related information display apparatus according to claim 1, wherein
the display control unit sets the early-morning blood pressure on a horizontal axis and the night-time blood pressure on a vertical axis.

6. A non-transitory computer-readable medium including a program for causing a computer to execute a method for displaying information related to blood pressure of a measurement subject on a display screen, the method comprising:
a first step of acquiring blood pressure data including at least early-morning blood pressure data measured in the morning and night-time blood pressure data measured at night in a certain period for the measurement subject; and
a second step of setting one of early-morning blood pressure and night-time blood pressure on a horizontal axis and the other on a vertical axis, and performing graph display of a point determined by the early-morning blood pressure data and the night-time blood pressure data on the display screen.
